(19) United States
(12) Patent Application Publication
(10) Pub. No.: US 2010/0131043 A1
(43) Pub. Date: May 27, 2010

(54) Endoluminal Implants For Bioactive Material Delivery

(76) Inventors: Jesus W. Casas, Brooklyn Park, MN (US); Amy C. Dearking, Rochester, MN (US); Rodolfo Padua, Richfield, MN (US); Molly Schiltgen, Lake Elmo, MN (US); Jeffrey P. Walker, Vancouver (CA)

Correspondence Address: K&L Gates LLP, 1900 MAIN STREET, SUITE 600, IRVINE, CA, 92614-7319, US

(21) Appl. No.: 12/324,193

(22) Filed: Nov. 26, 2008

(51) Int. Cl. A61F 2/44

(52) U.S. Cl. 623/1.15; 623/1.39; 623/1.23

(57) ABSTRACT

Disclosed herein are endoluminal implants for bioactive material delivery. The disclosed endoluminal implants come in the general forms of patches and sleeves and can deliver bioactive materials independently of other implantable medical devices such as stents.

### FIELD OF THE INVENTION

The present invention relates to endoluminal implants for bioactive material delivery. The endoluminal implants come in the general forms of patches and sleeves and can deliver bioactive materials independently of other implantable medical devices such as stents, grafts and other endoluminal devices.

### BACKGROUND OF THE INVENTION

Disease, injury, surgery or other disorders can lead to localized tissue damage. When bioactive materials are administered orally or parenterally to treat a local disorder, they often must be given in large amounts so that an effective amount of the bioactive material reaches the treatment site. These large amounts of administered bioactive materials can produce harmful side effects in other organs and areas of the body where treatment is not needed. Thus, one significant challenge in the medical and pharmaceutical industry has been to deliver an effective amount of a bioactive material locally, or regionally, targeting the treatment site without producing unwanted systemic side effects.

A prime example of a situation where local therapy is needed with bioactive materials that produce unwanted systemic side effects is in the prevention of complications following the placement of a stent within a lumen. For example, balloon angioplasty is a common procedure for widening a blocked lumen. During this procedure, a balloon is advanced to the site of a lumen narrowing and expanded at the site. Radial expansion of the affected lumen thus occurs in several different dimensions, and is related to the nature of the plaque narrowing the lumen. Soft, fatty plaque deposits are flattened by the balloon, while hardened deposits are cracked and split to enlarge the lumen. The wall of the vessel itself is also stretched when the balloon is inflated.

Generally, following a balloon angioplasty procedure, a stent is deployed at the treatment site in an attempt to prevent acute reclosure or reconstriction (hereinafter renarrowing) of the lumen. While approximately 1.8 million coronary blocked artery interventions are performed worldwide each year, however, renarrowing of the artery occurs in 10%-40% of these cases, typically within about six months of the initial procedure. This critical complication can lead to recurrence of severe symptoms and myocardial infarction, repeated interventions or more invasive bypass surgeries.

One approach to reducing the risk of lumen renarrowing following balloon angioplasty or a related procedure has been the introduction of bioactive material-eluting stents. Bioactive material-eluting stents can provide a benefit for local bioactive material delivery and a reduction in lumen renarrowing. However, stent placement addresses only the local vascular stenosis (atherosclerotic lesion) without treating the underlying disease that undermines the coronary vasculature. Progression of atherosclerotic lesions over time generates new localized vulnerable plaques with potential for provoking abrupt occlusion of the artery, and thus myocardial ischemia, if not treated with another angioplasty, stenting, or surgical revascularization (coronary artery bypass graft, CABG) procedure.

### SUMMARY OF THE INVENTION

The present disclosure provides endoluminal implants that deliver bioactive materials locally to a treatment site within a target organ or lumen or regionally downstream, distal to the implant site. In particular the disclosed endoluminal implants provide bioactive material delivery independent of stents or other implantable medical devices that provide physical lumen support. The endoluminal implants disclosed herein can be implanted within any organ or lumen where local or downstream regional therapy is needed. Endoluminal implants described herein can provide therapy in combination with, or adjuvant to, or in circumstances where classical devices, such as stents, are not a viable option (i.e., small diameter vessels, diffuse lesions, long lesions, lesions in small vessels, and lesions at bifurcated and/or curved vessels), or when stenting is not desired or possible.

In certain patch endoluminal implant embodiments, the patch adheres to the internal side of the organ wall or lumen through a mechanism selected from the group consisting of an active mechanism, a passive mechanism, and combinations thereof.

Treated lumens can include, without limitation, those within the cardiovascular system, the respiratory system, the gastrointestinal system, the urinary system, the lymphatic system, the biliary system, the reproductive system, etc. Bioactive materials released from the endoluminal implants can include, without limitation, drugs, proteins, genes, oligonucleotides, and combinations thereof and can also include, without limitation, anti-inflammatory agents, anti-proliferative agents, angiogenic agents, cell differentiation factors, proimmunogenic agents, anti-infective agents, pro-inflammatory agents, anti-angiogenic agents, agents with paracrine and/or endocrine activity, and combinations thereof.

In one embodiment, the endoluminal implant comprising an active fixation patch (FIGS. 1, 2A, 2B, 2C) is implanted via a minimally invasive procedure and is fixed to the intraluminal aspect of the target organ (i.e., artery, esophagous, vein, urether, urethra, respiratory conduits, Fallopian ductus, etc.) and either contacting the diseased tissue (for local effects) or proximal to the diseased/affected tissue (for regional downstream effects). The patch is selected from a variety of sizes and shapes depending on the target application and at least partially covers a portion of the inner lumen of the target organ for a temporary (absorbable) or permanent (non-absorbable) implantation. In another embodiment, the endoluminal implant is a passive fixation patch and, by virtue of its radially explandable rings (FIG. 3A, 3B), is deployed against the target lesion or target organ segment, from which the therapy is intended to be administered via the elution of bioactive materials or the production and release of the bioactive materials among others.

In yet another embodiment, an endoluminal device, an intraluminal sleeve (FIG. 4A, 5A, 5B) is provided wherein the endoluminal sleeve is an expandable tubular device, is deployed via a minimally invasive procedure and is contained in place by the device's radially expandable force (FIGS. 6A, 6B, 6C), constituting the source for temporary or a permanent delivery of bioactive materials with local or regional or systemic effect.

In one embodiment, an endoluminal implant is provided comprising a structural material and a bioactive material wherein the endoluminal implant is in the form of a sleeve or a patch and can be positioned within a lumen at a treatment site or proximal to the treatment site and wherein the endoluminal implant does not provide physical support of said lumen. In another embodiment, the structural material comprises at least one biocompatible metal or polymer having one or more of biodegradable, bioerodable, or bioadsorbable properties. In another embodiment, the polymer is biocompatible and one or more of biodegradable, bioerodable, or bioadsorbable. In another embodiment, the implant is smooth or porous.

In one embodiment, the implant is a sleeve and the sleeve comprises an incomplete tubular shape. In another embodiment, once the sleeve is positioned and deployed at the treatment site the diameter of the sleeve is larger than the lumen in which it is implanted.

In another embodiment, the implant is a patch and the patch adheres to the lumen through a mechanism selected from the group consisting of an active mechanism, a passive mechanism, and combinations thereof.

In another embodiment, the lumen is part of a system selected from the group consisting of the cardiovascular system, the respiratory system, the gastrointestinal system, the urinary system, the lymphatic system, and the biliary system.

In another embodiment, the bioactive material is selected from the group consisting of a drug, a protein, a gene, or functional constituents and combinations thereof. In another embodiment, the bioactive material is selected from the group consisting of an anti-inflammatory agent, an anti-proliferative agent, an angiogenic agent, a proimmunogenic agent, an anti-infective agent, a pro-inflammatory agent, an anti-angiogenic agent, agents with paracrine and/or endocrine effect, and combinations thereof.

### BRIEF DESCRIPTION OF THE FIGURES

FIG. 1 depicts an active-fixation endoluminal implant embodiment in patch form deployed within a tubular organ at a treatment site.

FIGS. 2A-2D depict unimplanted and cross-sectional implanted views of one embodiment of the active-fixation patch with magnified views of its porous intraluminal construct embodiment and its active fixation elements (FIGS. 2B-2D).

FIGS. 3A and 3B depict unimplanted and cross-sectional implanted views of a passive-fixation patch, respectively and show an embodiment with two expandable rings attached to the therapeutic patch. FIG. 3B depicts a likely relationship of the device within a luminal organ (e.g., vessel with an atherosclerotic lesion).

FIG. 4A depicts an endoluminal implant embodiment in sleeve form with a magnified view of bioactive material release (FIG. 4B).

FIGS. 5A and 5B depict sleeve embodiments deployed at treatment sites. FIG. 5A depicts an endoluminal sleeve proximal to diffuse lesions of tubular organs. FIG. 5B depicts the deployment of the endoluminal sleeve proximal to a vascular stent. In both cases, the therapy is released to provide effective therapy downstream, thus preventing worsening of the atherosclerotic condition and preventing the development of in-stent restenosis.

FIGS. 6A-6C depict one embodiment of a delivery mechanism for sleeve implants.

### DEFINITION OF TERMS

The following definition of terms is provided as a helpful reference for the reader. The terms used in this patent have specific meanings as they relate to the present invention. Every effort has been made to use terms according to their ordinary and common meaning. However, where a discrepancy exists between the common ordinary meaning and the following definitions, the definitions supersede the common usage.

Bioactive Materials: The term “bioactive material(s)” refers to any organic, inorganic, or living agent that is biologically active or relevant. For example, a bioactive material can be a protein, a polypeptide, a polysaccharide (e.g., heparin), an oligosaccharide, a mono- or disaccharide, an organic compound, an organometallic compound, or an inorganic compound. It can include a living somatic or stem cells, or senescent cell, bacterium, virus, DNA, RNAi, genes, or part thereof. It can include a biologically active molecule such as a hormone, a growth factor, a growth factor producing virus, a growth factor inhibitor, a growth factor receptor, an anti-inflammatory agent, an antimetabolite, an integrin blocker, or a complete or partial functional insense or antisense gene. It can also include a man-made particle or material, which carries a biologically relevant or active material. An example is a nanoparticle comprising a core with a drug and a coating on the core.

Bioactive materials can also include drugs such as chemical or biological compounds that can have a therapeutic effect on a biological organism. Bioactive materials include those that are useful for short-term therapy such as in stent restenosis and others and long-term therapy such as hormonal treatment, atherosclerosis therapy among others. Exemplary, non limiting examples include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers disclosed herein.

Bioactive materials also can include precursor materials that exhibit the relevant biological activity after being metabolized, broken-down (e.g., cleaving molecular components), or otherwise processed and modified within the body. These can include such precursor materials that might otherwise be considered relatively biologically inert or otherwise not effective for a particular result related to the medical condition to be treated prior to such modification.

Combinations, blends, or other preparations of any of the foregoing examples can be made and still be considered bioactive materials within the intended meaning herein. Aspects disclosed herein are directed toward bioactive materials can include any or all of the foregoing examples.

Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Lumen: The term “lumen” includes any area of an organism's body which transports substances and includes, but is not limited to, blood vessels of the cardiovascular system (arteries and veins), vessels of the lymphatic system, the intestinal tract (esophagus, stomach, the small and large intestines, and colon), the portal-caval system of the liver, the gall bladder and bile duct, the urinary system (ureters, bladder, and urethra), the respiratory system (trachea, bronchi, and bronchioles), and ducts and ductules connecting endocrine organs to other areas of the body.

Matrix: As used herein, the term “matrix” refers to structures within the disclosed endoluminal device which contain the bioactive materials.

Physical Support: As used herein, the phrase “physical support” refers to an implantable medical device, which provides a resisting force through direct contact with a lumen, the presence of which reduces the likelihood of lumen narrowing.

Treatment Site: The phrase “treatment site” includes any portion of a lumen that is intended to receive a beneficial or therapeutic effect of a bioactive material administered with the endoluminal implants described herein. For example, the treatment site can be locally, without limitation, a stenotic lesion in a blood vessel, a developing thrombus, a localized tumor, or the like or regionally without limitation, diffuse coronary atherosclerosis, or systemically without limitation, diabetes and other endocrine diseases.

### DETAILED DESCRIPTION

The present disclosure provides endoluminal implants that can deliver bioactive materials to a treatment site overcoming difficulties of the prior art. In particular, the disclosed endoluminal implants provide bioactive material delivery independently of stents or other implantable medical devices that provide physical support for the lumen. Divorcing the delivery of bioactive materials from other implanted devices removes a number of drawbacks associated with current treatment modalities including problems associated with the adherence of bioactive materials to implantable medical devices and the high costs associated with bioactive material-eluting stents. Further, the disclosed endoluminal implants can be implanted within any organ or lumen where local or downstream regional therapy is needed. Endoluminal implants described herein can be adjuvant or provide therapy in circumstances where classical devices, such as stents, are not a viable option (i.e., small diameter vessels, diffuse lesions, long lesions, lesions in small vessels, and lesions at bifurcated and/or curved vessels), or when stenting is not desired or possible.

Endoluminal implants according to the present disclosure, come in two general forms, “patches” and “sleeves,” each comprising and releasing at least one bioactive material. Patches can come in any number of appropriate shapes but generally will not extend more than about half way around the inner circumference of the treated lumen. Sleeves, on the other hand, do extend more than about half way around the inner circumference of the treated vessel and are more tubular in shape (although most sleeves according to the present disclosure do not comprise complete tubes but instead, comprise a break in material around the circumference of the implant). These two types of endoluminal implants deliver bioactive materials but do not provide physical lumen support. Representative embodiments of endoluminal implants, including patches and sleeves, are depicted in the accompanying figures.

Specifically, one embodiment includes the use of biomaterials such as metals and/or polymer(s), either as part of the structure of the device itself or applied on the device, for the controlled release of therapeutic agents. The endoluminal implant is in the form of a sleeve or a patch and can be positioned directly on or proximal to the targeted lesion (i.e., vulnerable plaque, abscess, localized cancer, localized hypertrophy, etc.) and the endoluminal implant does not provide physical support to the lumen. Endoluminal implants have body surfaces comprising biomaterials such as metals, polymers, etc, which in turn includes a multiplicity of micromachined porous with 5-100 μm diameter. These structures can be coated or assembled with smooth or porous biomaterials such as polymers that are biodegradable, bioerodable, and/or bioadsorbable to accommodate and facilitate the delivery of bioactive materials.

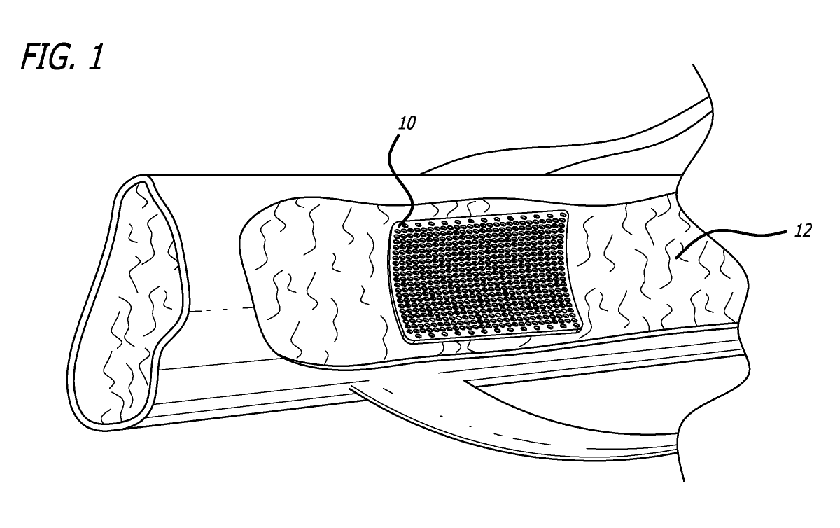

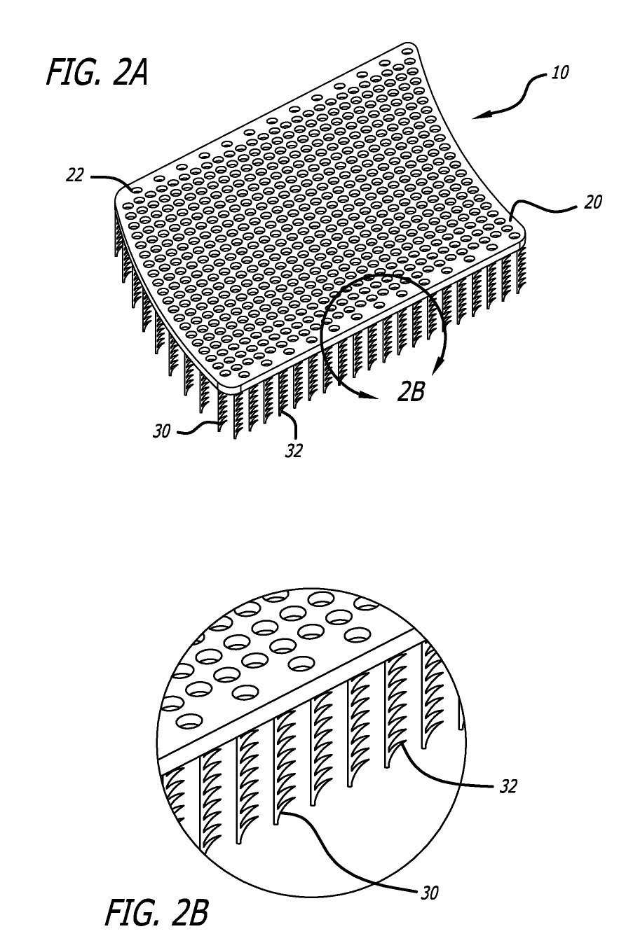

In one embodiment (FIG. 1), an endoluminal device is comprised of a therapeutic patch having an active fixation mechanism based on micro-hooks in peripheral needles (FIGS. 2A and 2B), which are introduced within the target tissue by compression during device deployment with the assistance of balloon catheters (FIG. 2C). FIG. 1 depicts a patch 10 implanted at a treatment site within a vessel lumen 12. FIG. 2A depicts this patch 10 before implantation at the treatment site. As can be seen, this depicted embodiment adopts an active attachment mechanism comprising a number of microhooks 32 on anchors 30, attached to patch body 20, that can engage the lumen wall or plaque or other material found at the treatment site (hereinafter collectively referred to as “material”). Patch body 20 includes passages 22 to allow passage of fluid and metabolic products to and from the patch therapeutic construct, and allows the deposition of host tissue for efficient endothelialization (FIG. 2C). The different vascular tissue layers are illustrated by structures 21 and 23 with 23 representing the innermost endothelial lining and 50 being a localized atherosclerotic lesion. When deployed at a treatment site within a lumen, the microhooks 32 of patch 10 engage material 50 at the treatment site to hold the patch in place at the treatment site (FIGS. 2B and 2C). The microhooks 32 are introduced into the target tissue by compression of patch 10 onto the target tissue, such as by a balloon catheter or other means of applying a force to the patch, and allowing microhooks 32 to engage the target tissue.

FIG. 2C, shows a section of an endoluminal device highlighting an embodiment which includes multilayered elements having therapeutic potential. From the upper aspect of FIG. 2D, the endoluminal device might include an anti-thrombotic treatment layer 22 (exposed to the blood stream), applied on a microporous surface or a semi-permeable layer 24 made of a biocompatible absorbable or non-absorbable materials, which allows selective transport of substances, a matrix 26 that contains a biological or synthetic elements to carry or provide reservoir for bioactive material/substances or living allogenic or autologous cells either for local delivery (FIG. 2D, 70) or for selective release of cell products (e.g., proteins) through the microporous layer (FIG. 2D, 60). The device also comprises a structural layer 28 which is preferred to have absorbable properties and is adapted to assist in maintaining the matrix elements at the treatment site. Layers 24 and 28 provide the boundary barriers for the releasable bioactive materials or for the biological tissue-engineered cell-based matrix contents, thus, the patch containing the therapeutic elements included in the matrix can be positioned for effective delivery at the target sites. In case of endoluminal devices having cell based components, the matrix can be comprised of biological extracellular components such as collagen, hyaluronic acid, elastin, and other proteins and/or glycoproteins and/or growth factors and/or intercellular mediators that facilitate or promote cell survival, proliferation and/or function of cells deposited within the matrix. Other examples, where delivery of bioactive agents is expected, the matrix layers components can be composed of polymeric reservoir and/or carriers, co-polymer and/or bioactive agent-polymer blends, and other combination types. The multiplicity of this combination and formulations are configured 1) to interface with a the unique histology and architecture of the target organ surface such as endothelium-smooth muscle cells in the cardiovascular system, epithelial histology in digestive tracts, etc. and 2) to interact with the intraluminal components (e.g., blood, urine, digestive fluids, air, bile fluid, etc,) and 3) to provide release of therapies at dosages and timing that are effective for a given target condition or pathophysiology. For example, to prevent in stent-restenosis, a short timed, acute, burst of appropriate anti-proliferative drug may be enough to prevent in-stent restenosis on bare (non-drug eluting) coronary stents. By contrast, a chronic sustained release of agents having anti-inflammatory agents and other metabolism-modulators may be needed for treating diffuse coronary atherosclerosis.

As can be seen in certain embodiments depicted in FIGS. 2C and 2D when deployed at a treatment site, patch 10 will curve so that pores 60 are created on the surface of patch 10 facing the lumen wall 23. When these pores 60 are created, bioactive materials 70 are released through them and become able to create an effect at the intended treatment site. This approach allows the delivery of bioactive materials at a treatment site without tying the delivery of the bioactive materials to a physical support providing implantable medical device such as a stent. The rate of release of bioactive materials can be controlled through a number of methods known to those of ordinary skill in the art. For example, bioactive material release can be controlled through, without limitation, adjusting pore size created upon implantation or including differentially permeable barrier layers to slow or accelerate bioactive material release. Likewise, a porous material can allows the passage of nutrients and products from cells entrapped or loaded within matrices as part of the endoluminal devices. Bioactive materials such as, but not limited to, vascular endothelial growth factor (VEGF), platelet derived growth factor (PDGF), tissue plasminogen activator (tPA), insulin, and dopamine can be released from biological cell-based microreactors contained in the patch matrix (FIG. 1D) or assembled on the luminal, parietal side of the devices, or combination thereof.

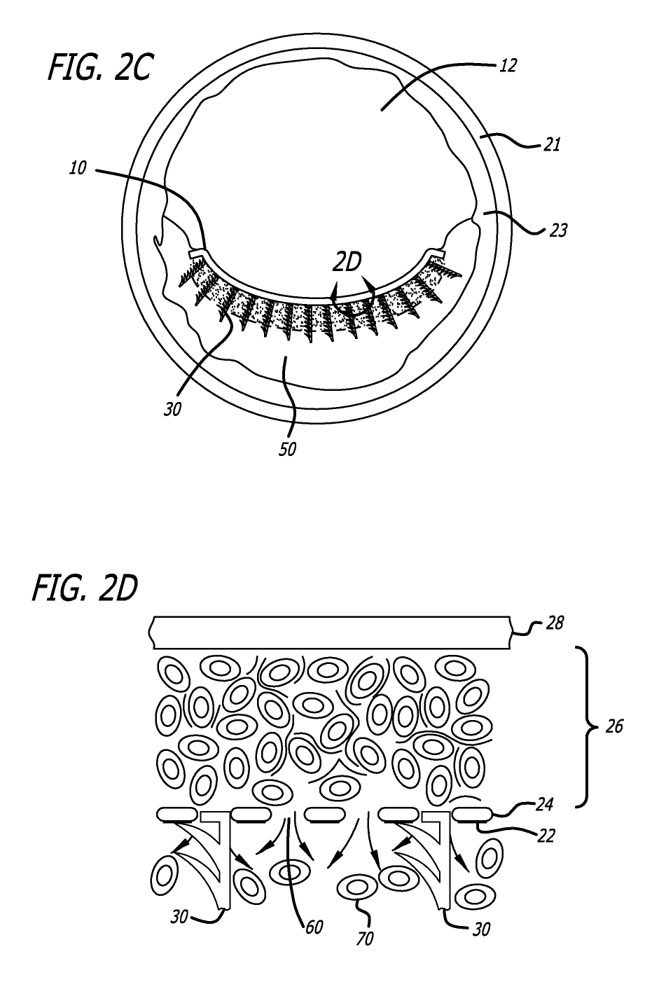

FIGS. 3A and 3B depict an alternative embodiment of a patch, wherein the patch adopts a passive fixation mechanism. In this depicted embodiment, the patch 300 has two circumferential expandable rings 310 attached to it. As will be understood by one of skill in the art, any appropriate number of rings 310 could be attached to patch 300 so long as the number and placement of these rings 310 are sufficient to hold patch 300 in place at the lumen treatment site. When patch 300 is positioned at a treatment site, rings 310 will expand and hold patch 300 in place by exerting sufficient force against the inner circumference of the lumen wall 23 (see FIG. 3B). Again, in this embodiment, when deployed at a treatment site, patch 300 curves so that the porous surface is placed on target site and the patch 300 with its therapeutic structures are left contacting the lumen wall 23 (FIG. 3B). These pores 360 for, bioactive materials 370 are released through them and become able to create an effect at the treatment site.

Here it should be noted that not all bioactive material release occurs through pores created on the surface of the endoluminal implants and the creation of pores upon implantation is not a required element of the disclosed devices. Bioactive material release can also occur through degradation of the polymer within the endoluminal implant or diffusion of bioactive materials in the presence or absence of created pores. Further, bioactive material release can also occur through any combination of the above methods.

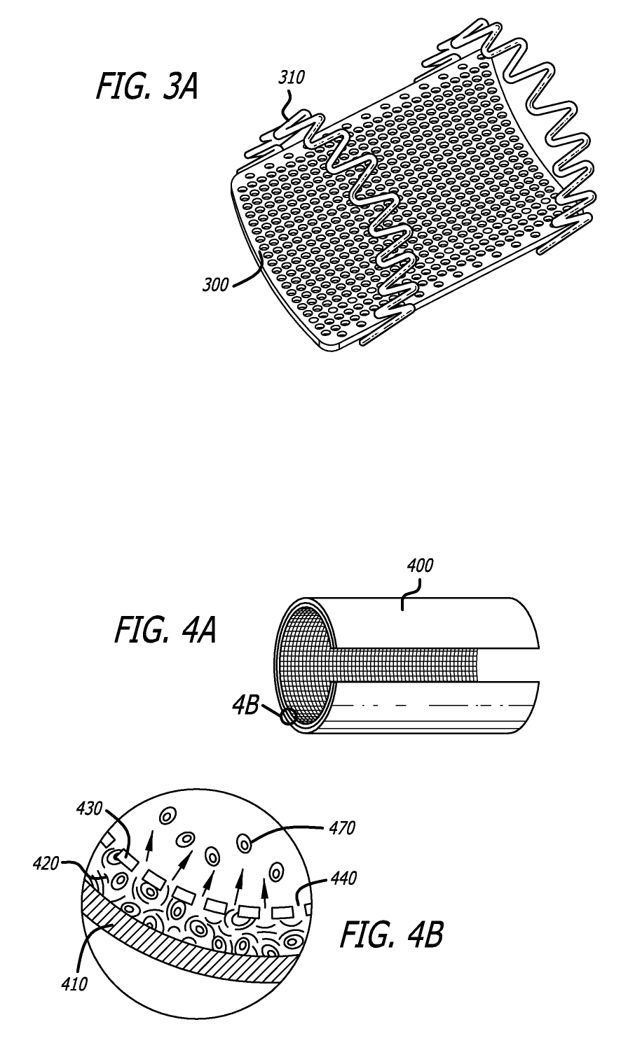

FIG. 4A depicts another embodiment, a “sleeve” endoluminal implant before implantation at a treatment site. Sleeves, as disclosed herein, are constructed with biological or synthetic absorbable biomaterials, generally arranged in an incomplete tubular shape, the tubular shape typically having a diameter slightly larger than the vessel or organ in which it is to be used. In one embodiment depicted in FIG. 4B, the sleeve comprises one or a pluralilty of semi-permeable layers 430 having structural openings or pores 440, allowing a selective transport of substances, a matrix reservoir 420 that contains a bioactive material 470 for local delivery through the microporous layer 430. The device also comprises a structural outerlayer layer 410 which is adapted to assist in maintaining the integrity of matrix containing the therapeutic components at the treatment site. In case of endoluminal devices, where delivery of bioactive agents is expected, the sleeve's matrix layer components can be composed of polymeric reservoir and/or carriers, co-polymer and/or bioactive agent-polymer blends, and other combination types. The multiplicity of these combination and formulations are configured 1) to interface with a the unique histology and architecture of the target organ surface such as endothelium-smooth muscle cells in the cardiovascular system, epithelial histology in digestive tracts, etc. and 2) to interact with the intraluminal components (e.g., blood, urine, digestive fluids, air, bile fluid, etc,) and 3) to provide release of therapies at dosages and timing that are effective for a given target condition or pathophysiology, 4) for a controlled and gradual reabsortion of the device during or following the delivery of therapy such that no remnants of the biodegradable sleeve are left. For example, to prevent in stent-restenosis, a endoluminal sleeve having a short timed, acute, burst of appropriate anti-proliferative drug or other agents may be enough to prevent in-stent restenosis on bare (non-drug eluting) coronary stents. By contrast, a chronic sustained release of agents having anti-inflammatory properties (e.g., statins, etc.) and other metabolism-modulators may be needed for treating diffuse coronary atherosclerosis.

As can be seen in the magnified view of FIG. 4A (FIG. 4B), when positioned and expanded at a treatment site, the depicted sleeve embodiment releases bioactive materials 470 (e.g., drugs, proteins, genes, oligonucleotides, cells, etc) into the area of blood flow. This embodiment is not limiting, however, and sleeve embodiments can be designed to release bioactive materials from either or both sides of the sleeve following expansion at a treatment site. As stated above, this bioactive material release can occur through pores created following expansion of the sleeve or through degradation of polymers, components of the device, within the endoluminal implant or through diffusion of the bioactive material in the presence or absence of created pores. If bioactive material release is not desired from a portion of an endoluminal implant (e.g., device's parietal aspect), non-permeable layers can be included to prevent bioactive material release from that portion of the implant. In one embodiment, the sleeve, once positioned and deployed at a treatment site can have a diameter that is slightly larger than the lumen in which it is implanted. This larger diameter provides enough force to maintain the position of the sleeve at or near the treatment site but does not provide enough force to provide physical support of the vessel. FIG. 4B, shows the multilayered aspect of the device depending on the agent to be delivered.

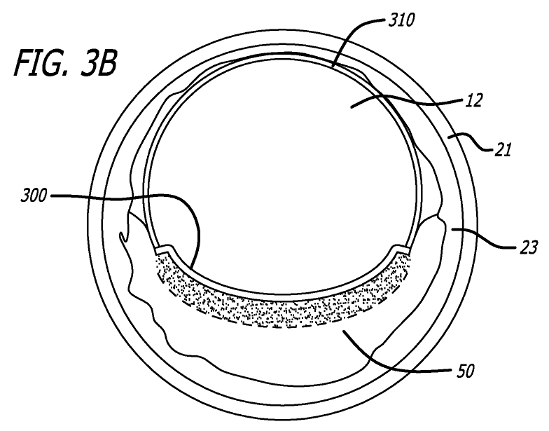

FIGS. 5A and 5B depict the sleeve depicted in FIG. 4 in two different treatment scenarios. In FIG. 5A, sleeve 400 is positioned within a lumen at an area proximal to a region requiring treatment due to the build up of atherosclerotic material 540. As the sleeve 400 releases bioactive materials 550, fluid flow 560 (in one embodiment blood flow) carries the bioactive materials 550 downstream to the treatment site. In this depicted embodiment, the endoluminal sleeve implant is used independently and as a stand alone treatment. As depicted in FIG. 5B, implants can also be used in conjunction with one or more stents. In this depicted embodiment, sleeve 400 is again positioned within a lumen at an area proximal to a region requiring treatment where a vascular stent has been deployed to maintain an adequate vessel lumen diameter compromised by the build up of material 540 (atherosclerotic lesion) and proximal to where stent 580 has been implanted. As the sleeve 400 releases bioactive materials 550, fluid flow 560 carries the bioactive materials 550 downstream to the treatment site. In this depicted embodiment, the endoluminal sleeve implant is used in conjunction with stenting to aid in the prevention of lumen renarrowing (a phenomenon also known as in-stent restenosis). As should now be understood by one of ordinary skill in the art, this approach allows the simultaneous delivery of bioactive materials at a stented treatment site. Likewise, these devices can be deployed proximal or on top, underneath bare (non-drug eluting) stents, thus delivering a temporary therapy during the risk period for incidence of in-stent restenosis.

While these two treatment scenarios are provided, they should be read as non-limiting examples of the treatment options available with the disclosed endoluminal implants. For example, in another embodiment when the endoluminal implants are used in conjunction with a stent, the endoluminal implant could be positioned at the same portion of the lumen as the stent either around the outer circumference or within the inner circumference of the stent. In yet other treatment scenarios, an endoluminal implant could be positioned in conjunction with or proximal to a bioactive material-eluting stent to provide a mechanism to increase the delivery of available bioactive materials. Further, while these treatment scenarios have generally been depicted and described using sleeves as the chosen endoluminal implant, patches are likewise appropriate for use in these treatment scenarios.

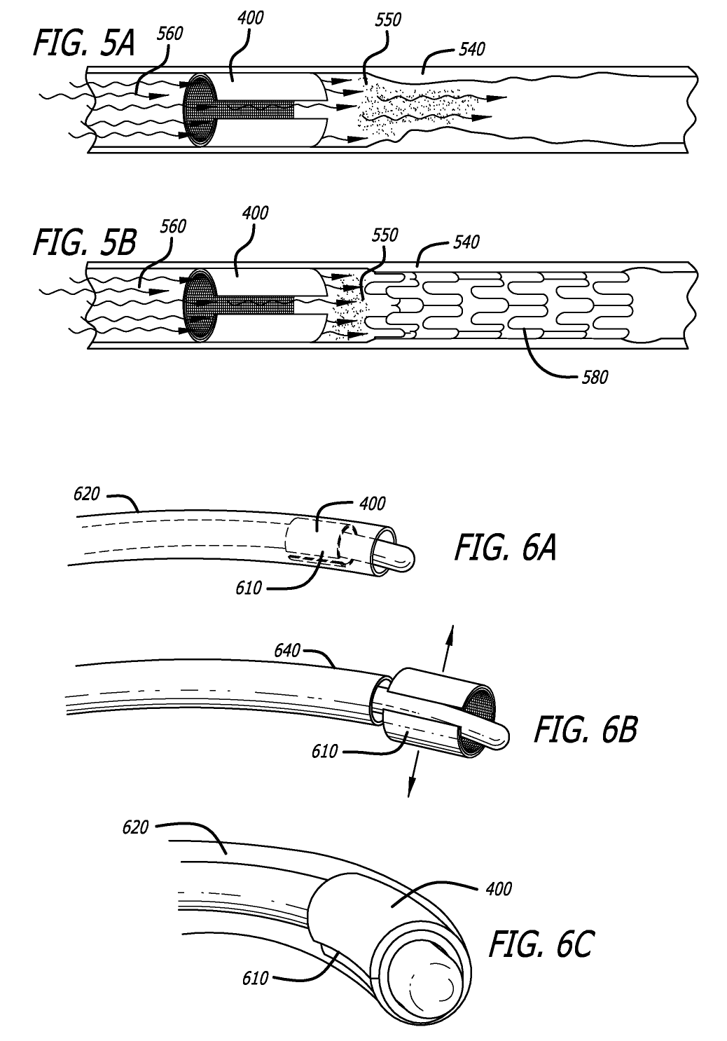

As stated, the endoluminal implants do not provide physical lumen support. In some embodiments, the described endoluminal implants can be provided “pre-loaded” into a delivery and deployment catheter. The distal end of one delivery and deployment catheter for a sleeve embodiment is depicted in FIGS. 6A-6C. As shown in FIGS. 6A and 6C, sleeve 400 can be assembled compressed, where lateral walls 610 slide over each other to reduce nominal diameter. Then, the sleeve 400 can be loaded within a delivery and deployment catheter 620, in which, in one non-limiting embodiment, a telescopic mechanism can hold the sleeve 400 in place. The catheter 620 can be advanced to a treatment site and by a retractable mechanism, an outer catheter layer 640 can release the sleeve 400 at or proximal to the treatment site (FIG. 6B). The sleeve 400, once released, can expand at the site of implantation and hold itself against the lumen wall.

The endoluminal implants can comprise flat, smooth or porous films that comprise, without limitation, synthetic or biologic, degradable, biocompatible polymers and chemistries. These polymer films or coatings can comprise various combinations of bioactive materials including, without limitation, drug-polymer, gene-polymer, protein-polymer, microparticles, liposomes, biodegradable blend-polymers carrying bioactive materials, and combinations thereof. When delivering genes by transfecting the adjacent tissue, the endoluminal implants can establish a tissue-based permanent local delivery system. The endoluminal implants can also be designed to deliver multiple therapies (i.e., one or more bioactive materials that are, individually or collectively, and without limitation, antithrombotic, proendothelialization, anti-inflammatory, anti-proliferative, anti-migration, angiogenic, pro-inflammatory, anti-angiogenic, pro-immunogenic, anti-infective, cell transfer, and combinations thereof). Furthermore the therapy can comprise cells, cellular products, genes, proteins, drugs, and combinations thereof.

Furthermore, the endoluminal implants can comprise a plurality of layers comprising different polymers and/or different bioactive materials in each layer. The polymer layers provide controlled-release of drugs. In one embodiment, one of the polymeric layers comprises an anti-thrombogenic agent. In another embodiment, the polymeric layer controls the release of drugs from other layers. In yet another embodiment, one or more polymeric layers comprise a porous or microporous material.

Structural portions of the endoluminal devices can be comprised of a plurality of materials which are selected depending upon the target organ, the time and dosage for active therapy. Thus, biocompatible metals (used in vascular stents, grafts, and other class III devices) can be used when permanent implantation of a therapeutic device is preferred. Absorbable or non absorbable polymers (bioredable, degradable devices) or copolymers, either naturally ocurring or synthetic biomaterials can be used as structural elements and/or in combination with the therapeutic elements. Thus, a combination of these elements can provide the desired bioactive response from herein disclosed endoluminal devices.

Endoluminal implants can be designed to deliver different bioactive materials sequentially, at an optimal rate and timing to downstream regions, or to the target tissue at treatment sites with which they interact. Bioactive material release can be short term to long term (from about 1 hour to years) depending on the therapeutic approach. Cells engrafting within the device or migrating to adjacent tissues can deliver sustained local or regional therapy for long periods of time (months to years) to tissue surfaces or organ regions in need of treatment. The endoluminal implants can provide local and/or regional delivery of bioactive materials including, without limitation, to the vascular, biliary, urinary, respiratory, gastrointestinal, lymphatic, and/or esophageal lumens. Pathologies susceptible to respond to this approach are primary or secondary, local or regional diseases of inflammatory, proliferative, thrombogenic, metabolic, or infectious etiology such as, without limitation, atherosclerosis, stenosis, ulcers, thrombosis, cancer, infection, etc. Additionally, there are infective pathologies that may be amenable to local therapies such as drug-resistant bacteria, fungal, and protozoan infections. The disclosed endoluminal implants may be an excellent methodology for promoting immune responses to various immunogens that are slowly released from the endoluminal implants. In certain embodiments, once the bioactive material is delivered from the device, the endoluminal implant will reabsorb.

Non-limiting examples of polymers that can be especially useful in accordance with the present disclosure include poly-lactic acid (PLA), poly-glycolic acid (PGA), polycarbonates, polyurethanes, polycapralactone, and polyorthoester. Other polymers that may be used as appropriate include, without limitation, rapidly bioerodible polymers such as, without limitation, poly[lactide-co-glycolide], polyanhydrides, and polyorthoesters whose carboxylic groups are exposed on the external surface as their smooth surface erodes. In addition, polymers containing labile bonds, such as, without limitation, polyanhydrides and polyesters can also be used. Representative natural polymers that can be used include, without limitation, proteins, such as zein, modified zein, casein, gelatin, gluten, serum albumin, or collagen, and polysaccharides, such as, without limitation, cellulose, dextrans, polyhyaluronic acid, polymers of acrylic and methacrylic esters, and alginic acid. Representative synthetic polymers that can be used include, without limitation, polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, and copolymers thereof. Synthetically modified natural polymers that can be used include, without limitation, alkyl celluloses, hydroxyalkyl celluloses, cellulose ethers, cellulose esters, and nitrocelluloses. Other polymers that can be used include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, and polyvinylphenol. Representative bioerodible polymers include polylactides, polyglycolides and copolymers thereof, poly(ethylene terephthalate), poly(butic acid), poly(valeric acid), poly(lactide-co-caprolactone), poly[lactide-co-glycolide], polyanhydrides, polyorthoesters, and blends and copolymers thereof.

These described polymers can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif. or else synthesized from monomers obtained from these suppliers using standard techniques.

Various adaptations and modifications of the embodiments can be made and used without departing from the scope and spirit of the present disclosure which can be practiced other than as specifically described herein. The above description is intended to be illustrative, and not restrictive. The scope of the present invention is to be determined only by the claims.

The terms and expressions which have been employed herein are used as terms of description and not of limitation, and there is no intention in the use of such terms and expressions of excluding equivalents of the features shown and described, or portions thereof, it being recognized that various modifications are possible within the scope of the present invention claimed. Moreover, any one or more features of any embodiment of the present invention can be combined with any one or more other features of any other embodiment of the present invention, without departing from the scope of the present invention.

Unless otherwise indicated, all numbers expressing quantities of ingredients, properties such as molecular weight, reaction conditions, and so forth used in the specification and attached claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the present invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the present invention otherwise claimed. No language in the specification should be construed as indicating any non-claimed element essential to the practice of the present invention.

Groupings of alternative elements or embodiments of the present invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these certain embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above cited references and printed publications are individually incorporated herein by reference in their entirety.

In closing, it is to be understood that the embodiments of the present invention disclosed herein are illustrative of the principles of the present invention. Other modifications that may be employed are within the scope of the present invention. Thus, by way of example, but not of limitation, alternative configurations of the present invention may be utilized in accordance with the teachings herein. Accordingly, the present invention is not limited to that precisely as shown and described.

What is claimed is:

1. An endoluminal implant comprising a structural material and a bioactive material wherein said endoluminal implant is in the form of a sleeve or a patch and can be positioned within a lumen at a treatment site or proximal to said treatment site and wherein said endoluminal implant does not provide physical support of said lumen.

2. An endoluminal implant according to claim 1 wherein the structural material comprises at least one biocompatible metal or polymer having one or more of biodegradable, bioerodable, or bioadsorbable properties.

3. An endoluminal implant according to claim 2 wherein said polymer is biocompatible and one or more of biodegradable, bioerodable, or bioadsorbable.

4. An endoluminal implant according to claim 1 wherein said implant is smooth or porous.

5. An endoluminal implant according to claim 1 wherein said implant is a sleeve and said sleeve comprises an incomplete tubular shape.

6. An endoluminal implant according to claim 3 wherein once said sleeve is positioned and deployed at said treatment site the diameter of said sleeve is larger than the lumen in which it is implanted.

7. An endoluminal implant according to claim 1 wherein said implant is a patch and said patch adheres to said lumen through a mechanism selected from the group consisting of an active mechanism, a passive mechanism, and combinations thereof.

8. An endoluminal implant according to claim 1 wherein said lumen is part of a system selected from the group consisting of the cardiovascular system, the respiratory system, the gastrointestinal system, the urinary system, the lymphatic system, and the biliary system.

9. An endoluminal implant according to claim 1 wherein said bioactive material is selected from the group consisting of a drug, a protein, a gene, or functional constituents and combinations thereof.

10. An endoluminal implant according to claim 1 wherein said bioactive material is selected from the group consisting of an anti-inflammatory agent, an anti-proliferative agent, an angiogenic agent, a proimmunogenic agent, an anti-infective agent, a pro-inflammatory agent, an anti-angiogenic agent, agents with paracrine and/or endocrine effect, and combinations thereof.
